(19) United States
(12) Patent Application Publication
(10) Pub. No.: US 2022/0217286 A1
(43) Pub. Date: Jul. 7, 2022

(54) CORRECTION OF HORIZON TILT IN AN ENDOSCOPIC IMAGE

(71) Applicant: Asensus Surgical US, Inc., Durham, NC (US)

(72) Inventors: Caleb T. Osborne, Durham, NC (US); Evan Mix, Durham, NC (US)

(21) Appl. No.: 17/567,084

(22) Filed: Dec. 31, 2021

Related U.S. Application Data
(60) Provisional application No. 63/133,409, filed on Jan. 3, 2021.

(51) Int. Cl. H04N 5/262; H04N 7/18; G06T 7/70; G06T 3/60; H04N 5/232; B25J 9/12; A61B 1/00; A61B 5/06

(52) U.S. Cl. CPC H04N 5/2628; H04N 7/183; G06T 7/70; G06T 3/60; H04N 5/23299; B25J 9/12; H04N 2005/2255; A61B 1/00045; A61B 1/0016; A61B 5/067; G06T 2207/10068; G06T 2207/10016; A61B 1/000095

(57) ABSTRACT

In a system and method for correcting horizon line incongruity in a display of an image captured by an endoscope. The system determines an axial rotation of an angled endoscope while the endoscope is capturing video images of a surgical site. The capture images are displayed in real time on an image display, and, more particularly, are displayed as rotated by an equal degree to the amount of axial rotation, and in an opposite direction to the determined axial rotation. This avoids the undesirable shift of the horizon line on the viewer's monitor that typically occurs during axial rotation of an angled scope.

This application claims the benefit of U.S. Provisional Application No. 62/133,409, filed Jan. 3, 2021.

### BACKGROUND

An angled endoscope is one in which the optical axis of the scope is angularly offset from the longitudinal axis of the scope. It is a tool commonly used in laparoscopic surgery. The angled scope allows for visualization of target anatomy and provides additional options for trocar port and instrument placement.

Pan and zoom motions of an angled scope can be challenging, as the view is not centered on the axis of the endoscope shaft. Additionally, the angle of the scope is typically oriented downward, which makes visualizing port sites challenging. Typical practice when the surgeon wishes to view a port site is to roll the scope, thus rolling the angle about the endoscope axis. This motion has an effective panning motion, as the angle of the scope provides a view to the left or right of the original view. However, the rotation of the scope creates an incorrect horizon which can make instrument manipulation challenging.

This application describes features enabling digital rotation of an on-screen image to compensate for rotation of the camera.

### BRIEF DESCRIPTION OF THE DRAWINGS

FIG. 1 schematically shows a system according to the disclosed invention;

FIG. 2 is a sequence of images showing a body cavity, and possible orientations of a view box of a laparoscopic camera positioned to capture images of that cavity. The images at the lower left illustrate how the view boxes would be displayed on an endoscope display using conventional techniques. The images at the right illustrate the process of detecting rotation of the scope and adjusting the display to account for the rotation.

FIG. 3 illustrates an image of a body cavity, and shows viewing boxes in three rotational orientations.

FIG. 4 schematically depicts rotation orientation of the viewing boxes for three rotational positions of the endoscope.

FIG. 5 illustrates a comparison of the endoscope view versus the monitor view for each of the rotational orientations shown in FIG. 4.

### DETAILED DESCRIPTION

System

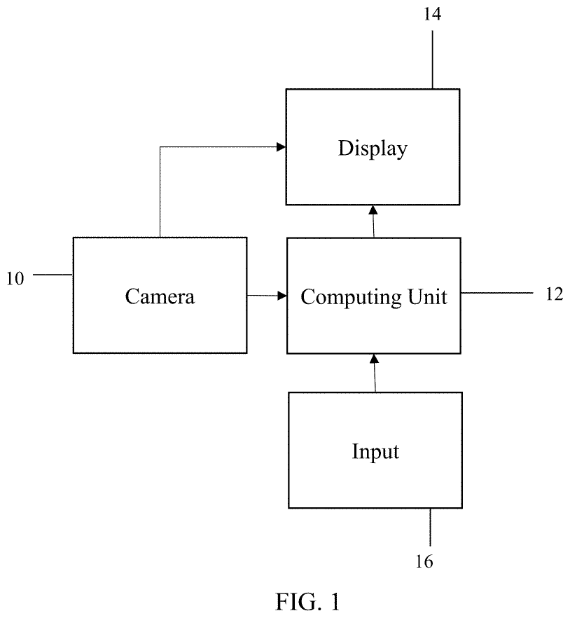

A system useful for performing the disclosed methods is depicted in FIG. 1. The system may optionally be used in conjunction with a robot-assisted surgical system in which surgical instruments are maneuvered within the surgical space using one or more robotic components (e.g., robotic manipulators that move the instruments and/or camera, and/or robotic actuators that articulate joints, or cause bending, of the instrument or camera shaft).

The system may comprise an angled endoscopic or laparoscopic camera 10, one or more computing units 12, and a display 14. The camera 10 is one suitable for capturing images of the surgical site within a body cavity. It may be a 3D or 2D endoscopic or laparoscopic camera. The angle may be a fixed angle, or the scope may be one moveable to one or more angled positions.

The computing unit 12 is configured to receive the images/video from the camera. It may also be configured to receive data from other sources 16, where the data allows the computing unit to receive data corresponding to changes in the rotational orientation of the camera around its longitudinal axis, as described in this application. The data may be input from which the computing unit can determine or derive the changes in the rotational orientation, or it may be input providing the computing unit with the degree of change in the rotation orientation.

In some embodiments, the input may be received from components of the camera. For example, an inertial measurement unit (IMU) on the camera might provide such data. As a second example, where the camera includes integral electromechanical features for rotating the camera or a distal portion of the camera about the longitudinal axis, the input representing changes in the rotational orientation of the imaging head is obtained from the corresponding components of the endoscope. In embodiments where the camera is rotated using a robotic manipulator, the system may be configured so that the computing unit receives kinematic information from the robotic manipulator or associated robotic components.

Still other embodiments might use computer vision applied to features captured in the endoscope image to determine the degree to which the scope has been rotated about its longitudinal axis. Where the system uses an image processing/computer vision algorithm to detect whether the scope is an angled scope that is rotating, the algorithm is used to track motion of areas detected in the image data. The system analyses whether the image data shows both a panning motion as well as rotation. Image processing algorithm analysis is used to determine if the rotation is resulting in a panning motion as well as a rotation. Pure rotation would suggest that an angled scope is not in use. Pan and rotation suggests an angled scope is in use. The image processing detects landmarks in the endoscopic view and then maintains those landmarks at a fixed horizon.

An algorithm stored in memory accessible by the computing unit is executable to, depending on the particular application, use the input data to perform one or more of the functions described with respect to the disclosed embodiments.

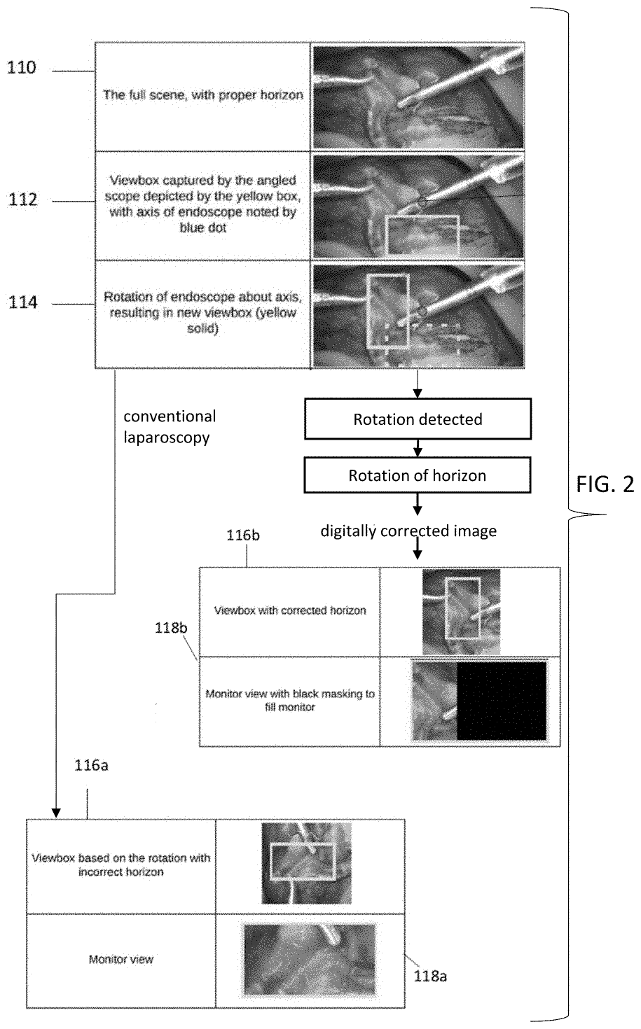

Referring to FIG. 2, block 110 shows a surgical site, and block 112 shows a border around the view of the surgical site that would be captured (“viewbox”) by the angled endoscope. It is also how the image would be displayed on the image display. The circle or dot D above the border aligns with the longitudinal axis of the scope.

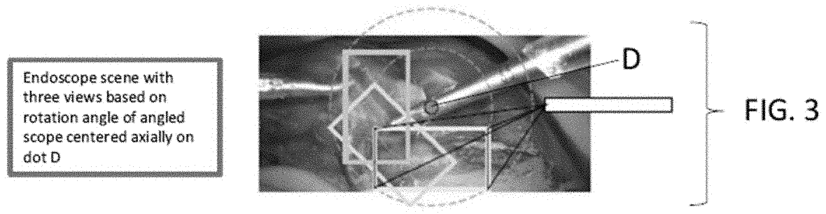

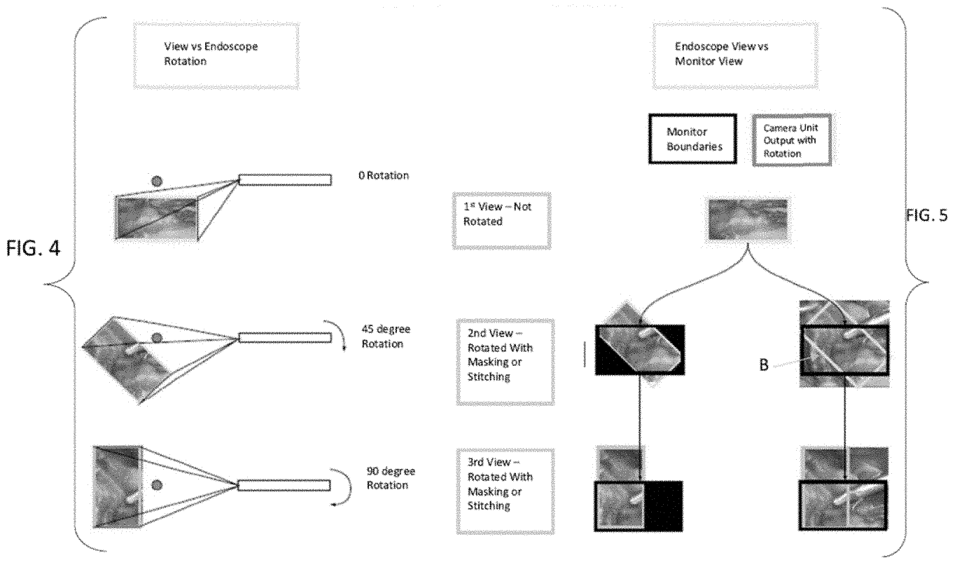

Block 114 represents the scene after the camera has been rotated 90 degrees. The solid border represents the current viewbox, and the dashed border represents the pre-rotation viewbox from Block 112. In conventional laparoscopy, the viewbox would be displayed in the horizontal/landscape orientation as illustrated in Block 116a, with the solid border representing the current viewbox, rotated to a landscape horizon. Block 118a shows how that current viewbox would thus be conventionally presented on the image display without the currently disclosed features. See FIG. 3, which shows the scene at the surgical site. The three rectangles with the light borders show the endoscope views with the angled camera at 0 degrees, 45 degrees and 90 degrees rotation, respectively, around its longitudinal axis (which is aligned with the small circle D that is shown). This is also schematically depicted in the three schematic drawings of the endoscope and the captured view shown in FIG. 4. Using conventional methods, each of these viewboxes would be displayed horizontally, thus making its horizon incorrect relative to the scene.

Method

In use, the system determines the amount by which the scope has been rotated. In this determining step, the system may derive the amount from received input, or it may receive input specifying the amount. In either case the determining step uses at least one of (i) image data from the camera image, (ii) telemetry data from a robotic manipulator that maneuvers the scope, or from electrometrical components that rotate a distal part of the scope, and (iii) an inertial momentum unit (IMU) sensor on the camera. If the scope is determined to have rotated, the system automatically corrects for the degree of horizon line incongruity that would occur as a result of the rotation by rotating the endoscopic image (for display) by an equal and opposite amount as the amount of derived or input rotation. The result is a “digital pan” where the scene appears to pan left or right but the only endoscope motion is an axial rotation. By maintaining the horizon line, it is easier for the surgeon to maintain cognitively oriented with respect to the image, and so instrument manipulation within the surgical site is simpler and more natural.

In FIG. 5, the endoscope image view and the displayed or “monitor” view are shown for each of the three scope angles represented in FIG. 4. In each case, the endoscope image view is depicted with a light border, while the boundaries of the monitor view are depicted with a dark border. The greater the degree of roll of the camera to the left, the further the rotated endoscopic image appears to the left of the monitor view, and the greater the degree of roll to the right, the further to the right it appears.

At zero degrees rotation, the image view fills the monitor view. As the endoscope is rotated and the resulting image is counter-rotated to provide a consistent horizon, the resulting image will not fill the monitor if a camera system with a rectangular image output is used. In this case, two methods of presenting the image may be used. In one method, shown on the left side of FIG. 5, the image processing unit may mask areas of the screen for which no image data has been received for display. In the other method, shown on the right side of FIG. 5, the image processing unit may stitch previous frames to the edges of the live, real time, (rotated) frame using image processing technique. A border B around the endoscope view may be displayed as an overlay on the monitor, serving as a clear indicator that the stitched portion of the image (i.e., the portion outside border B) is no not a live image but it is based on previous frames prior to the rotation of the angled scope.

Whereas a typical roll motion of the scope allows an angled scope to center on a point lateral to center, such a motion also results in the undesirable shift of the horizon line on the viewer's monitor. The solution described in this application allows correction of this horizon tilt, thereby delivering on a surgeon's need to roll to angle scope to a point desired, while simultaneously rotating the image on the monitor to provide an image with the correct horizon line.

What is claimed is:

1. A method of correcting horizon line incongruity in a display of an endoscope image, comprising the steps of:
determine an axial rotation of an angled scope while said endoscope is capturing video images of a surgical site;
displaying in real time the captured images on an image display, wherein the displayed captured images are displayed as rotated by an equal degree to the amount of axial rotation, and in an opposite direction to the determined axial rotation.

2. The method of claim 1, wherein determining the axial roll comprises applying image process to the captured video images to detect and/or measure the axial rotation.

3. The method of claim 1, wherein determining the axial roll includes receiving kinematic information from a robotic component operable to axially roll the endoscope.

4. The method of claim 3, wherein the robotic component is a robotic manipulator operable to maneuver the endoscope.

5. The method of claim 3, wherein the robotic component is an electromechanical manipulator integral with the endoscope.

6. The method of claim 1, wherein determining the axial roll includes receiving input from an inertial measurement unit carried by the endoscope.

7. The method of claim 1, further including displaying masking on the portion of the image display left unfilled by the rotated real time image.

8. The method of claim 1, further including, on the portion of the image display left unfilled by the rotated real time image, displaying portions of a non-real time image of the surgical site stitched to the real time image.

9. A system for correcting horizon line incongruity in a display of an image captured by an endoscope, comprising:
an image display; and
a computing unit configured to receive input, and a memory accessible by the computing unit, the memory including a program executable to,
determine an axial roll of an angled scope while said endoscope is capturing video images of a surgical site;
display in real time the captured images on an image display, wherein the displayed captured images are displayed as rotated by an equal degree to the amount of axial rotation, and in an opposite direction to the determined axial rotation.

10. The system of claim 9, wherein the computing unit is configured to receive input in the form of video images captured by the endoscope, and wherein the program is executable to determine the axial roll by applying image processing to the captured video images to detect and/or measure the axial roll.

11. The system of claim 9, wherein the computing unit is configured to receive input in the form of kinematic information from a robotic component operable to axially roll the endoscope, and wherein the program is executable to determine the axial roll using the kinematic information.

12. The system of claim 11, wherein the robotic component is a robotic manipulator operable to maneuver the endoscope.

13. The system of claim 11, wherein the robotic component is an electromechanical manipulator integral with the endoscope.

14. The system of claim 9, wherein the computing unit is configured to receive input in the form of input from an inertial measurement unit carried by the endoscope, and wherein the program is executable to determine the axial roll using the input from the inertial measurement unit.

15. The system of claim 9, wherein the program is executable to display masking on a portion of the image display left unfilled by the rotated real time image.

16. The system of claim 9, wherein the program is executable to, on the portion of the image display left unfilled by the rotated real time image, display portions of a non-real time image of the surgical site stitched to the real time image.
